(19) United States
(12) Patent Application Publication
(10) Pub. No.: US 2010/0129805 A1
(43) Pub. Date: May 27, 2010

(54) SLC1A1 ANTIPSYCHOTIC DRUG RESPONSE MARKERS

(76) Inventors: Wai Lun Alan Fung, Willowdale (CA); James Lowery Kennedy, Toronto (CA)

Correspondence Address: WINSTON & STRAWN LLP;PATENT DEPARTMENT, 1700 K STREET, N.W., WASHINGTON, DC, 20006, US

(21) Appl. No.: 12/514,060

(22) PCT Filed: Oct. 31, 2007
(86) PCT No.: PCT/CA2007/001943

§ 371 Date: Jan. 26, 2010

Related U.S. Application Data
(60) Provisional application No. 60/865,101, filed on Nov. 9, 2006.

(51) Int. Cl. C12Q 1/68

(52) U.S. Cl. 435/6

(57) ABSTRACT

The invention relates to methods for predicting a subject's response to antipsychotic drug treatment comprising the steps of obtaining a biological sample from the subject, and determining the presence or absence of one or more polymorphisms in the SLC1A1 gene of the subject, wherein the presence of the one or more polymorphisms indicates that the subject's response to antipsychotic drug treatment. The invention also provides for kits for performing these methods.

### FIELD OF INVENTION

The present invention relates to diagnostics associated with symptom response to antipsychotics.

### BACKGROUND OF THE INVENTION

The glutamatergic dysfunction hypothesis of schizophrenia (SCZ) is based on the observation that phencyclidine, an N-methyl-D-aspartate (NMDA) glutamate receptor antagonist, gives rise to a SCZ-like psychosis (Kim et al, 1980; Jentsch and Roth, 1999). Glutamate is synthesized in the cytoplasm and stored in synaptic vesicles. Following its exocytotic release, glutamate activates ionotropic and metabotropic glutamate receptors for fast excitatory neurotransmission and slower modulatory effects on transmission, respectively. To terminate glutamate action, Na+-dependent high affinity glutamate transporters (excitatory amino acid transporters: EAATs) located on plasma membrane of neurons and glial cells rapidly remove glutamate from the extracellular space (Shigeri et al, 2004). As such, downregulation of EAATs may increase glutamate availability in the extracellular space—and may be associated with decreased SCZ symptoms. To date, five types of EAATs (EAATs 1-5) have been identified. Among these, EAAT3 (encoded by the gene SLC1A1) is predominantly localized to neurons. The SLC1A1 gene (OMIM#133550), located on chromosome 9p24, consists of 12 exons and is about 97 kb in size. It is highly expressed within cerebral cortex, striatum and thalamus (Kanai and Hediger, 2004).

It has been estimated that clozapine is an effective treatment for at least 70% of schizophrenic (SCZ) patients refractory or intolerant to typical antipsychotics (APs) (Meltzer, 1997). Schmitt et al (2003) demonstrated in a rat study that gene expressions of EAAT3 in the cingulate cortex, infralimbic cortex, hippocampal CA1 & CA2, and striatum, are significantly downregulated by clozapine treatment over 6 months. As such, genetic factors affecting the function of SLC1A1 gene may affect EAAT3 function and in turn, clozapine response.

### SUMMARY OF THE INVENTION

The present invention relates to diagnostics associated with symptom response to antipsychotics.

According to the present invention, there is provided a method of predicting a subject's response to antipsychotic drug treatment comprising,

a) obtaining a biological sample from the subject;

b) determining the presence or absence of one or more polymorphisms in the SLC1A1 gene of the subject, wherein the presence of said one or more polymorphisms indicates the subject's response to the antipsychotic drug treatment.

The present invention also provides a method as defined above, wherein the one or more polymorphisms comprise one or more polymorphisms as defined by SEQ ID NOs:1, 2, 3, 4, 5 or a combination thereof.

Also contemplated by the present invention is a method as defined above wherein the one or more polymorphisms comprise one or more polymorphisms having a sequence that exhibits between about 90% and 100% sequence identity with SEQ ID Nos 1, 2, 3, 4, 5 or a combination thereof.

The present invention also provides a method as defined above, wherein said response is the change in positive symptoms, negative symptoms or both. In an embodiment, which is not meant to be limiting, the response is the change in positive symptoms. In an alternate embodiment, the response is the change in negative symptoms. Further, the positive symptoms may comprise one or more of delusions, hallucinations, disorganized speech, disorganized behavior, and catatonic behavior and the negative symptoms comprise one or more symptoms that reflect a diminution or loss of normal function. In a preferred embodiment, the response is relative to a psychiatric rating scale, for example, but not limited to the Brief Psychiatric rating scale (BPRS), the positive symptom subscale (BPOS), the negative symptom subscale (BNEG), or a combination thereof. Other scales are also contemplated.

The present invention also provides a method as defined above, wherein the antipsychotic drug comprises a drug that affects dopamine signaling. The drug may comprise clozapine, trifluoperazine, thioridazine, haloperidol, haloperidol decanoate, thiothixene, chlorpromazine, fluphenazine, loxapine, perphenazine, perphenazine decanoate, perphenazine-amitriptyline, acetophenazine, molindone, mesoridazine, fluphenazine decanoate, methotrimeprazine, risperidone, aripiprazole or a combination thereof. In a preferred embodiment, the antipsychotic drug comprises clozapine or a combination therapy comprising clozapine.

The present invention also provides a method as defined above, wherein the subject is a Caucasian subject or an African-American subject.

The present invention also provides a method as defined above, wherein the subject exhibits psychotic symptoms, Schizophrenia symptoms, Schizoaffective disorder symptoms or a combination thereof. In a preferred embodiment, the subject is diagnosed as having a disorder with psychotic symptoms, Schizophrenia or Schizoaffective disorder.

Also contemplated is a method as defined above wherein the sample is selected from the group consisting of blood, saliva, spinal fluid, brain biopsy, cultured cells obtained from the subject, stool, urine, autopsy samples, or frozen sections. Preferably, the sample is blood.

Also provided is a method as defined above, wherein the step of determining is performed by PCR analysis, sequencing, 5′ exonuclease fluorescence assay, probe hybridization or a combination thereof.

The present invention also provides a method of predicting a subject's response to antipsychotic drug treatment comprising,

a) obtaining a biological sample from the subject;

b) determining the presence or absence of one or more polymorphisms selected from the group consisting of SEQ ID NO:1, SEQ ID NO:2, SEQ ID NO:3, SEQ ID NO:4, SEQ ID NO:5, or any combination thereof, wherein,

the presence of the C allele of the rs301434 polymorphism (SEQ ID NO: 5) in Caucasians predicts improvement in overall psychosis symptoms, for example, but not limited to a decrease in mean BPRS % scores,

the presence of the C allele of the rs301434 polymorphism (SEQ ID NO:5) in Caucasians and African-Americans combined predicts improvement in negative symptoms, for example, but not limited to a decrease in mean BNEG % scores,

the G/A genotype of the rs1980943 polymorphism (SEQ ID NO:1) in African-Americans predicts improvement in negative symptoms, for example, but not limited to a decrease in mean BNEG % scores,

To the haplotype 1-2 in SNP window 3-4 (the G allele of the rs2228622 polymorphism (SEQ ID NO:3) in combination with the C allele of the rs301430 polymorphism (SEQ ID NO:4)); the haplotype 2-1 in SNP window 4-5 (the C allele of the rs301430 polymorphism (SEQ ID NO:4) in combination with the T allele of the rs301434 polymorphism), and haplotype 1-2-1 in SNP window 3-4-5 (the G allele of the rs2228622 polymorphism (SEQ ID NO:3) in combination with the C allele of the rs301430 polymorphism (SEQ ID NO:4) and the T allele of the rs301434 polymorphism (SEQ ID NO:5)) in African Americans are each associated with worsening of negative symptoms, for example, but not limited to an increase in mean BNEG % scores,

the haplotype 2-1 in SNP window 3-4 (the A allele of the rs2228622 polymorphism (SEQ ID NO:3) in combination with the T allele in the rs301430 polymorphism (SEQ ID NO:4)), haplotype 1-1 in SNP window 4-5 (the T allele in the rs301430 polymorphism in combination with the T allele in the rs301434 polymorphism (SEQ ID NO:5) and haplotype 2-1-1 in SNP window 3-4-5 (the A allele in the rs2228622 polymorphism (SEQ ID NO:3), in combination with the T allele in the rs301430 polymorphism (SEQ ID NO:4) and the T allele in the rs301434 polymorphism (SEQ ID NO:5) in African Americans are each associated with improvement in negative symptoms, for example, but not limited to a decrease in mean BNEG % scores,

the haplotype 2-2-1 in SNP window 1-2-3 (the A allele in the rs1980943 polymorphism (SEQ ID NO:1) in combination with the C allele in the rs3780415 polymorphism (SEQ ID NO:2), and the G allele in the rs2228622 polymorphism (SEQ ID NO:3)) and haplotype 2-1-1 in SNP window 2-3-4 (the C allele of rs3780415 (SEQ ID NO:2) in combination with the G allele of the rs2228622 polymorphism (SEQ ID NO:3) and the T allele of the rs301430 polymorphism (SEQ ID NO:4) in Caucasians are both independently associated with worsening of negative symptoms, for example, but not limited to an increase in mean BNEG % score,

the haplotype 2-2-1 in SNP window 1-2-3 (allele A of the rs1980943 polymorphism (SEQ ID NO:1) in combination with allele C of the rs3780415 polymorphism (SEQ ID NO:2) and allele G of the rs2228622 polymorphism (SEQ ID NO:3)) in Caucasians and African Americans combined is associated with worsening of negative symptoms, for example, but not limited to an increase in mean BNEG % score, and;

the haplotype 1-1-1 in SNP window 1-2-3 (allele G of the rs1980943 polymorphism (SEQ ID NO:1) in combination with allele T of the rs3780415 polymorphism (SEQ ID NO:2) and allele G of the rs2228622 polymorphism (SEQ ID NO:3)) for Caucasians and African Americans combined, is associated with improvement of positive symptoms, for example, but not limited to a decrease in mean BPOS % score.

The present invention also contemplates a kit comprising,

a) one or more primers to amplify a nucleotide sequence that comprises the polymorphism as defined in SEQ ID NO:1, SEQ ID NO:2, SEQ ID NO:3, SEQ ID NO:4, SEQ ID NO:5 or a combination thereof;

b) one or more probes that hybridize to SEQ ID NOs:1, 2, 3, 4, or 5, over a region of nucleotides comprising the polymorphic site, wherein said probe hybridizes to a particular variant of the polymorphism at the polymorphic site.

c) one or more reagents and/or products comprising buffers, nucleotides, DNA amplifying enzymes, or any combination thereof;

d) one or more reagents, components and/or products for genotyping the polymorphisms of SEQ ID NO: 1, 2, 3, 4, 5, or a combination thereof,

e) one or more reagents, components and/or products for performing a DNA sequencing reaction that determines the sequence of a polynucleotide comprising SEQ ID NO: 1, 2, 3, 4, 5, or a combination thereof;

f) one or more instructions for using the components as described herein, practicing the methods as described herein, interpreting the data obtained from practicing the methods, or any combination thereof;

g) a scale, reference or the like that may be used to test, diagnose, monitor or determine a baseline of symptoms for a subject, or

h) any combination or subcombination of a) through g).

The kit as defined above may also comprise the Brief Psychiatric Rating Scale (BPRS), positive symptom subscale (BPOS), negative symptom subscale (BNEG), or a combination thereof.

This summary of the invention does not necessarily describe all features of the invention.

### DETAILED DESCRIPTION

The present invention relates to diagnostics associated with symptom response to antipsychotics.

The following description is of a preferred embodiment.

The present invention provides a genetic marker that may be used to predict a subject's response to antipsychotic drug therapy. As described in more detail below, specific polymorphisms in the SLC1A1 gene may be used to predict a subject's response to antipsychotic drug therapy.

The study described in the examples and as referred to herein and throughout investigated the effect of 5 single nucleotide polymorphisms (SNPs) across the SLC1A1 gene on antipsychotic response in two distinct schizophrenic populations refractory or intolerant to conventional antipsychotics. The subjects included 97 patients with DSM-III-R or DSM-IV diagnoses of schizophrenia—genotyped by 5′-exonuclease fluorescence assays. Within each population, genotype, allele+/−, and haplotype groups were compared on Brief Psychiatric Rating Scale (BPRS) overall, positive (BPOS) and negative symptom subscales (BNEG) at 6 months using analysis of variance. Results indicate that rs301434 was significantly associated with total BPRS % score change (P=0.0125) in Caucasians. The same SNP was significantly associated with BNEG % score change (P=0.049) for Caucasians and African-Americans combined. In African-Americans, rs1980943 was significantly (P=0.0167) associated with BNEG % score change. Specific haplotypes in SNP windows rs2228622-rs301430, rs301430-rs301434, and rs2228622-rs301430-rs301434 were also significantly associated with increase or decrease of BNEG % score in African Americans (P<0.05). Also identified are other haplotypes that were significantly associated with increase in BNEG % score in Caucasians, and haplotypes significantly associated with increase in BNEG % score and decrease in BPOS % score in Caucasians and African-Americans combined.

According to the present invention, there is provided a method of predicting a subject's response to antipsychotic drug treatment comprising,
a) obtaining a biological sample from the subject;
b) determining the presence or absence of one or more polymorphisms in the SLC1A1 gene of the subject, wherein the presence of said one or more polymorphisms indicates the subject's response to antipsychotic drug treatment.

By the term “one or more polymorphisms in the SLC1A1 gene” it is meant one or more polymorphisms in the nucleotide sequences as defined by:

rs1980943; SNP #1; SEQ ID NO: 1:
AGAGTCAGTTGGTGCATACATGTCC[A/G]GCATTTGCAAGAGAGGGTCT
GTGTT
rs3780415; SNP #2; SEQ ID NO: 2:
TGAGGTGAAAAAAGCAAATAAATGC[C/T]TTTACCTAAAATAAAGAGCT
AGCAG
rs2228622; SNP #2; SEQ ID NO: 3:
CAGGCAGCACCCCTGAAGTCAGTAC[A/G]GTGGATGCCATGTTAGATCT
CATCA
rs301430; SNP #4; SEQ ID NO: 4:
GTGCAACAATCAACATGGATGGGAC[C/T]GCGCTCTATGAAGCAGTGGC
AGCGG
rs301434; SNP #5; SEQ ID NO: 5:
GGCAAGGACTTGTCTCCAGAAAGCA[C/T]TGTAGGTGTGGCCTCCAGCT
TATCC

wherein the polymorphic site in each sequence is shown in bold, underlined brackets in relation to the nucleotide sequences upstream and downstream thereof. The present invention also contemplates one or more polymorphisms in one or more nucleotide sequences in the SLC1A1 gene which comprise between about 90% and 100% sequence identity, for example, but not limited to 90%, 91%, 92%, 93%, 94%, 95%, 96%, 97%, 98%, 99%, 99.5%, 99.9% or 100% sequence identity with SEQ ID NO:1, SEQ ID NO:2, SEQ ID NO:3, SEQ ID NO:4, or SEQ ID: NO:5, and wherein the sequence also comprises the respective polymorphisms as shown above in bold underlined brackets. For example, but not to be considered limiting in any manner, the first nucleotide shown in SEQ ID NO:5 is a “G”. The present invention is meant to include a sequence that is substantially identical to SEQ ID NO:5 but that comprises a “C” at position number 1, as the variant nucleotide sequence exhibits more than 90% sequence identity with SEQ ID NO:5 and comprises the polymorphism shown in bold underlined brackets.

To determine whether a nucleic acid exhibits similarity or a percentage identity with the sequences presented herein, oligonucleotide alignment algorithms may be used, for example, but not limited to a BLAST (GenBank URL: www.ncbi.nlm.nih.gov/cgi-bin/BLAST/, using default parameters: Program: blastn; Database: nr; Expect 10; filter: default; Alignment: pairwise; Query genetic Codes: Standard(1)), BLAST2 (EMBL URL: http://www.embl-heidelberg.de/Services/index.html using default parameters: Matrix BLOSUM62; Filter: default, echofilter: on, Expect:10, cutoff: default; Strand: both; Descriptions: 50, Alignments: 50), or FASTA, search, using default parameters. Polypeptide alignment algorithms are also available, for example, without limitation, BLAST 2 Sequences (www.ncbi.nlm.nih.gov/blast/bl2seq/bl2.html, using default parameters Program: blastp; Matrix: BLOSUM62; Open gap (11) and extension gap (1) penalties; gap x_dropoff: 50; Expect 10; Word size: 3; filter: default).

An alternative indication that two nucleic acid sequences are substantially identical is that the two sequences hybridize to each other under moderately stringent, or preferably stringent, conditions. Hybridization to fitter-bound sequences under moderately stringent conditions may, for example, be performed in 0.5 M NaHPO₄, 7% sodium dodecyl sulfate (SDS), 1 mM EDTA at 65° C., and washing in 0.2×SSC/0.1% SDS at 42° C. for at least 1 hour (see Ausubel, et al. (eds), 1989, Current Protocols in Molecular Biology, Vol. 1, Green Publishing Associates, Inc., and John Wiley & Sons, Inc., New York, at p. 2.10.3). Alternatively, hybridization to filter-bound sequences under stringent conditions may, for example, be performed in 0.5 M NaHPO₄, 7% SDS, 1 mM EDTA at 65° C., and washing in 0.1×SSC/0.1% SDS at 68° C. for at least 1 hour. Hybridization conditions may be modified in accordance with known methods depending on the sequence of interest (see Tijssen, 1993, Laboratory Techniques in Biochemistry and Molecular Biology—Hybridization with Nucleic Acid Probes, Part I, Chapter 2 “Overview of principles of hybridization and the strategy of nucleic acid probe assays”, Elsevier, New York). Generally, but not wishing to be limiting, stringent conditions are selected to be about 5° C. lower than the thermal melting point for the specific sequence at a defined ionic strength and pH.

As will be appreciated by a person of skill in the art, the term “SLC1A1 gene” is meant to include, without limitation, nucleotide sequences in the coding region(s) of the gene, including introns and exons, nucleotide sequences in the upstream regions of the coding sequence, for example, but not limited to in the promoter region or 5′ untranslated regions, nucleotide sequences in the regions downstream of the coding sequence, for example, but not limited to 3′ untranslated regions. In this regard, further information concerning the location of the polymorphisms described herein may be found throughout the application.

By the term “predicting a subject's response” it is meant predicting the change in positive symptoms, negative symptoms or both associated with a disorder, for example, but not limited to schizophrenia, schizoaffective disorder or a disorder that comprises psychotic symptoms. Without wishing to be limiting, positive symptoms can comprise one or more of delusions, hallucinations, disorganized speech, disorganized behavior, and catatonic behavior while negative symptoms can comprise one or more symptoms that reflect a diminution or loss of normal function. In a preferred embodiment, the “response” is graded or determined in relation to a psychiatric rating scale, for example, the Brief Psychiatric rating scale (BPRS) overall, the positive symptom subscale (BPOS), the negative symptom subscale (BNEG), or a combination thereof. However, other psychiatric rating scales may also be used.

In an embodiment of the present invention, but without wishing to be limiting in any manner, the method as described herein may be employed to determine a subject's response to antipsychotic medication, wherein at the time of screening the subject appears healthy. This information may be important when screening subjects that have a familial history of schizophrenia or other disorders with psychotic symptoms, even though at the time of screening, the subject may have little or no symptoms of disease. Knowledge of how a subject is likely to respond to antipsychotic medication may be useful in developing treatment regimens if for example, the subject later develops schizophrenia or psychotic symptoms and requires treatment.

Treatment-refractoriness is defined according to criteria in Kane et al 1988, which is herein incorporated by reference. This is a rigorous set of research criteria designed specifically to study the efficacy of a novel agent for treatment-refractory schizophrenia. The criteria include:

Clinical History: Absence of periods of good functioning in the preceding five years during treatment with antipsychotics at doses equal to or greater than chlorpromazine 1000 mg/day. During this time the patient must have received at least two different chemical classes of antipsychotic for six-weeks or more with no episodes of significant relief;

Cross-sectional: The patient must have a Brief Psychiatric Rating Scale (BPRS) score>45 (standard 18-item version, i.e. absent=1 through very severe=7) with item scores>4 (moderate severity) for two of the following four items: conceptual disorganization, suspiciousness, hallucinatory behavior, and unusual thought content and a Clinical Global Impression (CGI) score>4 (moderately ill);

Prospective Clinical Response: Failure to decrease BPRS score by 20% or below 35, or decrease CGI score to 3 (mildly ill) after a six-week prospective trial of haloperidol up to 60 mg/day.

By the term “antipsychotic drug” it is meant any drug, pharmaceutical, natural product, composition or the like that may be employed to prevent and/or treat psychosis, schizophrenia, schizoaffective disorder, disruptive behavior, disorganized thinking, symptoms of mania, or any combination thereof in a subject. Antipsychotic medication may comprise, but is not limited to, drugs that affect dopamine signaling, for example, drugs that bind reversibly or irreversibly to one or more dopamine receptors, drugs that act as competitive or non-competitive inhibitors to down-regulate dopamine receptor signaling, or that block the dopamine D2 receptor (see for example Seeman et al, 1976 and Seeman et al, 2005). Without wishing to be limiting, antipsychotic drugs comprise clozapine, trifluoperazine, thioridazine, haloperidol, haloperidol decanoate, thiothixene, chlorpromazine, fluphenazine, loxapine, perphenazine, perphenazine decanoate, perphenazine-amitriptyline, acetophenazine, molindone, mesoridazine, fluphenazine decanoate, methotrimeprazine, risperidone, aripiprazole or a combination thereof. In a preferred embodiment, the antipsychotic drug comprises clozapine or a combination therapy comprising clozapine.

In an embodiment of the present invention, subjects from any ethnic race, age, gender or medical condition may be tested to predict the subject's response to antipsychotic drug treatment. In this regard, a healthy subject or a subject that does not have any symptoms of a disease or medical condition may be tested to determine response to antipsychotic medication. In this way, if treatment is ever needed, a proper drug and/or treatment regimen may be selected and/or administered to the subject. In a preferred embodiment, a subject diagnosed with a disorder with one or more psychotic symptoms, schizophrenia, or schizoaffective disorder is tested to predict response to antipsychotic drug therapy, for example, but not limited treatment with clozapine or combination therapy comprising clozapine.

As described above, but without wishing to be limiting in any manner, the subject that is tested preferably comprises an individual with one or more psychotic symptoms, schizophrenia symptoms, schizoaffective order symptoms or a combination thereof, for example, but not limited to as described in DSM-IV which is hereby incorporated by reference. The psychotic symptoms may comprise positive symptoms such as, but not limited to distortions or exaggerations of inferential thinking (i.e. delusions), perception (i.e. hallucinations), language and communication (disorganised speech) and behavioral monitoring (grossly disorganized or catatonic behavior) or any combination thereof. Further, the positive symptoms may comprise distinct dimensions, for example, psychotic dimensions including, but not limited to delusions and hallucinations and disorganization dimensions including, but not limited to disorganized speech and behavior. As described previously, it is also contemplated that the symptoms may comprise one or more negative symptoms, for example, but not limited to symptoms that reflect a diminution or loss of normal function. Further, the subject may exhibit a combination of both positive and negative symptoms. In a preferred embodiment of the invention, the subject that is tested has been diagnosed or is suspected of having Schizophrenia or Schizoaffective Disorder.

Any tissue sample may be used for genotyping SLC1A1 polymorphisms, including but not limited to, blood, saliva, spinal fluid, brain biopsy, cultured cells obtained from the subject, stool, urine, autopsy samples, or frozen sections taken for histologic purposes. In a preferred embodiment, the subject is a mammal, preferably a human. In certain examples, blood is obtained from a subject for assaying with respect to SLC1A1 polymorphisms. As an example, but without wishing to be limiting in any manner, venous blood is obtained from a subject using standard venipuncture techniques.

The DNA of the subject may be tested for the presence or absence of the single nucleotide polymorphism (SNPs) by any suitable technique known in the art, Representative techniques that may be employed include without limitation PCR analysis, sequencing, 5′exonuclease fluorescence assay, probe hybridization or a combination thereof.

Polymorphisms may be genotyped using conventional techniques. For example, PCR using primers incorporating fluorescent probes is one suitable technique. Further, but not wishing to be considered limiting, primers having appropriate sequences upstream and downstream of the polymorphic site may be used to amplify the nucleotide regions comprising the polymorphisms.

Single nucleotide polymorphism (SNP) analysis is useful for detecting differences between alleles of the SLC1A1 gene. As described above, various methods exist in the art for genotyping nucleotide sequences including, but not limited to 5′exonuclease assays, sequencing, and the like. All such methods are meant to be encompassed herein. Further, various real-time PCR methods that can be used to detect SNPs, including, e.g., Taqman or molecular beacon-based assays (U.S. Pat. Nos. 5,210,015; 5,487,972; and PCT WO 95/13399) are useful to monitor for the presence or absence of a SNP. Still other SNP detection methods are known in the art, including, without limitation, DNA sequencing, sequencing by hybridization, dot blotting, oligonucleotide array (DNA Chip) hybridization analysis.

Applied Biosystems, Inc (Foster City, Calif.) has developed several aspects of SNP genotyping technology. In one well-used protocol, PCR amplification of a desired SNP region is conducted using targeting primers, including two allele-specific fluorogenic probes, each consisting of a different fluorescent reporter dye and a fluorescent quencher. Prior to PCR, proximity of the quencher to the fluorphore causes fluorescence resonance energy transfer (FRET), reducing the fluorescence from the reporter dye. During PCR, the 5′ nuclease activity of Taq digests the allele-specific probe bound to the region of the SNP, releasing the fluorescent dye from the quencher and allowing generation of a fluorescence signal.

The method of obtaining a sample and analyzing its DNA is not critical to the present invention and any methods may be used (e.g. Ausubel, et al. (eds), 1989, Current Protocols in Molecular Biology, Green Publishing Associates, Inc., and John Wiley & Sons, Inc., New York, at p. 2.10.3, or Maniatis et al., in Molecular Cloning (A Laboratory Manual), Cold Spring Harbor Laboratory, 1982, p. 387 389). For example, which is not to be considered limiting in any manner, DNA may be extracted using a non-enzymatic high-salt procedure. Alternatively, the DNA may be analyzed in situ. Other methods of DNA analysis that are known to persons skilled in the art may also be used.

Several scientific collaborations have attempted to identify and/or classify SNPs for genomes of several species including Homo sapiens, Arabidopsis thaliana, Caenorhabditis elegans, Ficedula albicollis, Ficedula hypoleuca, Gallus gallus, Mus musculus, Pan troglodytes, Plasmodium falciparum, and Rattus norvegicus. For example, the HapMap project attempts to determine the common patterns of human DNA sequence variation (haplotypes). SNP genotypes, recombination rates and other types of information may be browsed at or downloaded from the HapMap website (www.hapmap.org). SNPs are typically identified by location within a nucleotide sequence, or by a database assigned reference SNP ID number (“rs” number). In addition to HapMap, SNPs may be searched using various other resources. For example, individual rs numbers of the SNPs that are known to be located in a sequence of interest may be obtained by conducting a Blast search at the UCSC Genome Bioinformatics Web Page (www.genome.ucsc.edu). Conversely, sequence and scientific literature information associated with a given rs number may be obtained by searching the dbSNP of the Entrez SNP search option provided by the NCBI web page (www.ncbi.nlm.nih.gov).

In an embodiment of the present invention, which is not meant to be considered limiting, there is provided a method of predicting a subject's response to antipsychotic drug treatment comprising,

a) obtaining a biological sample from the subject;

b) determining the presence or absence of one or more polymorphisms selected from the group consisting of SEQ ID NO:1, SEQ ID NO:2, SEQ ID NO:3, SEQ ID NO:4, SEQ ID NO:5, or any combination thereof, wherein,

the presence of the C allele of the rs301434 polymorphism (SEQ ID NO: 5) in Caucasians predicts improvement in overall psychosis symptoms, for example, but not limited to a decrease in mean BPRS % scores,

the presence of the C allele of the rs301434 polymorphism (SEQ ID NO:5) in Caucasians and African-Americans combined predicts improvement in negative symptoms, for example, but not limited to a decrease in mean BNEG % scores,

the G/A genotype of the rs1980943 polymorphism (SEQ ID NO:1) in African-Americans predicts improvement in negative symptoms, for example, but not limited to a decrease in mean BNEG % scores,

the haplotype 1-2 in SNP window 3-4; the haplotype 2-1 in SNP window 4-5, and haplotype 1-2-1 in SNP window 3-4-5 in African Americans are each associated with worsening of negative symptoms, for example, but not limited to an increase in mean BNEG % scores,

the haplotype 2-1 in SNP window 3-4, haplotype 1-1 in SNP window 4-5 and haplotype 2-1-1 in SNP window 3-4-5 in African Americans are each associated with improvement in negative symptoms, for example, but not limited to a decrease in mean BNEG % scores,

the haplotype 2-2-1 in SNP window 1-2-3 and haplotype 2-1-1 in SNP window 2-3-4 in Caucasians are both independently associated with worsening of negative symptoms, for example, but not limited to an increase in mean BNEG % score,

the haplotype 2-2-1 in SNP window 1-2-3 in Caucasians and African Americans combined is associated with worsening of negative symptoms, for example, but not limited to an increase in mean BNEG % score, and;

the haplotype 1-1-1 in SNP window 1-2-3 for Caucasians and African Americans combined, is associated with improvement of positive symptoms, for example, but not limited to a decrease in mean BPOS % score.

To further clarify the nomenclature used by persons of skill in the art, by the phrase “the haplotype 1-2 in SNP window 3-4” it is meant the combined presence of the G allele at the polymorphic site in SNP 3; rs2228622 (SEQ ID NO:3) and the C allele at the polymorphic site in SNP 4; rs301430 (SEQ ID NO:4). Similarly, by the phrase “haplotype 1-2-1 in SNP window 3-4-5” it is meant the combined presence of the presence of the G allele at the polymorphic site in SNP 3; rs2228622 (SEQ ID NO:3), the C allele at the polymorphic site in SNP 4; rs301430 (SEQ ID NO:4), and the T allele at the polymorphic site in SNP 5; rs301434; SEQ ID NO:5). Further information regarding the alleles and haplotypes may be found in the Examples, Tables 1-4 and the entire specification.

The present invention also contemplates products and kits for practicing the methods of the present invention. For example, in respect of a kit, the kit may comprise:

a) one or more primers to amplify a nucleotide sequence that comprises the polymorphism as defined in SEQ ID NO:1, SEQ ID NO:2, SEQ ID NO:3, SEQ ID NO:4, SEQ ID NO:5 or a combination thereof;
b) one or more probes that hybridize to SEQ ID NOs:1, 2, 3, 4, or 5, over a region of nucleotides comprising the polymorphic site, wherein said probe hybridizes to a particular variant of the polymorphisms shown at the polymorphic site. Without wishing to be limiting in any manner, the probes may be labeled with an appropriate group, for example, a fluorescent tag, fluorophore, radioactive label or the like. Further, the one or more probes may be attached covalently or physically associated with a support for example, but not limited to a bio-chip, array, slide, multiwell plate, bead or the like. In an embodiment, which is not meant to be limiting in any manner, the probes may comprise an array of nucleic acids.
c) one or more reagents and/or products including, but not limited to, one or more buffers for performing PCR or probe hybridization, or any step in such as process as would be known to a person of skill in the art, one or more DNA amplifying enzymes, or any combination thereof;
d) one or more reagents, components and products for genotyping the polymorphisms as described herein, including, but not limited to those used in exonuclease assays, nucleotide sequencing, or any combination thereof;
e) one or more reagents, components or products for performing a DNA sequencing reaction that determines the sequence of a nucleotide sequence comprising SEQ ID NO: 1, 2, 3, 4, 5, or a combination thereof.
f) one or more sets of instructions for using the components as described herein, practicing the methods of the present invention as described herein, interpreting the data obtained from practicing the methods of the present invention or any combination thereof, and
g) a scale, reference or the like that may be used to test, diagnose, monitor or determine a baseline of symptoms for a subject. For example, but not wishing to be limiting, the scale may comprise the Brief Psychiatric Rating Scale (BPRS) overall, positive symptom subscale (BPOS), negative symptom subscale (BNEG), or a combination thereof. Other scales are also contemplated.

The present invention will be further illustrated in the following examples.

### EXAMPLES

### Example 1

### Effect of 5 Single Nucleotide Polymorphisms (SNPs) Across the SLC1A1 Gene on Clozapine (CZ) Response After 6 Months Treatment in Two Distinct Schizophrenic Populations (Caucasian and African-American) Refractory or Intolerant to Conventional Antipsychotics.

Methods

Subjects

97 patients (73 Caucasians and 24 African-Americans) with DSM-III-R or DSM-IV diagnoses of SCZ assessed from Case Western Reserve University, Cleveland, Ohio.

No significant differences were observed between Caucasians and African-Americans in terms of gender ratio (P=0.234) or mean age (P=0.561). Almost all subjects met criteria for treatment refractoriness or intolerance to typical antipsychotic therapy as defined by Kane et al (1988). Patients underwent a washout period of 2 to 4 weeks during which, unless clinically necessary, they received no medications before starting clozapine (CZ). CZ treatment was continued for a minimum of 6 months during which patients were evaluated prospectively. CZ blood levels were monitored throughout course of treatment to ascertain compliance,

Outcome Measures

Treatment response is evaluated as a percentage (%) score change from baseline using the 18-item Brief Psychiatric Rating Scale (BPRS), a 4-item positive symptom subscale (BPOS) and 3-item negative symptom subscale (BNEG)—after 6 months of clozapine treatment. % score change=(6 months score−baseline score)/Baseline score. A negative value of % score change in each scale would indicate an improvement in symptoms measured by that particular scale.

Laboratory Methods

Blood samples were collected from clinical sites and sent to the Centre for Addiction and Mental Health (CAMH) in Toronto, ON, Canada—where genotyping of patients' DNA was performed by 5′-exonuclease fluorescence assays. The 5 informative SNPs (minor allele frequency>20%) spanning SLC1A1 were chosen based on location (see Table 1)—with 2 in coding regions and 3 from different intronic regions and different haplotype blocks. Laboratory staff was blind to the psychiatric ratings.

TABLE 1
SLC1A1 SNPs used in this study

| SNP# | SNP | Alleles (1/2) | Location |
|---|---|---|---|
| 1 | rs1980943 | G/A | Intron 1 |
| 2 | rs3780415 | T/C | Intron 2 |
| 3 | rs2228622 | G/A | Exon 4 |
| 4 | rs301430 | T/C | Exon 10 |
| 5 | rs301434 | T/C | Intron 10 |

Statistical Methods

Within each ethnic population, individual SNP analyses of % score changes (continuous data) were performed using Analysis of Variance (ANOVA) in STATA ver7.0. Haplotype analysis of unphased quantitative data was performed using QTPHASE (Dudbridge, 2003).

Results

No significant deviation from Hardy-Weinberg equilibrium was observed for any of the 5 SNPs studied in either Caucasian or African-Americans. As well, possible confounding factors of age and gender did not have a significant influence on treatment outcome in either population. Among these SNPs, no haplotype block was observed in any of the 2 populations.

BPRS, BPOS and BNEG % change score distributions of genotype groups were compared against each other for each of the 5 SNPs, In Caucasians, SNP 5 (rs301434) was found to be significantly associated with total BPRS % change (P=0.0125). For the total population (Caucasians and African-Americans combined), SNP 5 was significantly associated with BNEG % score change (P=0.049). In the African-American population, the SNP 1 (rs1980943) was significantly (P=0.0167) associated with BNEG % score change. Tables 2a-c show the score changes associated with these 3 scenarios.

TABLE 2a
Mean BNEG % score change in rs1980943 in African-Americans
(P = 0.0167 overall)

| Genotype | Mean BNEG change* | N | SD |
|---|---|---|---|
| G/G | 0.067 | 9 | 0.690 |
| G/A | −0.378 | 6 | 0.347 |
| A/A | 3.000 | 2 | 4.243 |

TABLE 2b
Mean BPRS % score change in rs301434 in
Caucasians (P = 0.0125 overall)

| Genotype | Mean BPRS change* | N | SD |
|---|---|---|---|
| T/T | 1.310 | 7 | 4.057 |
| T/C | −0.310 | 35 | 0.320 |
| C/C | −0.248 | 23 | 0.356 |

TABLE 2c
Mean BNEG % score change in rs301434 in Caucasians &
African-Americans combined (P = 0.049 overall)

| Genotype | Mean BNEG change* | N | SD |
|---|---|---|---|
| T/T | 0.636 | 11 | 1.868 |
| T/C | −0.136 | 46 | 0.735 |
| C/C | −0.115 | 22 | 0.619 |

Haplotype analyses performed on the BPRS/BPOS/BNEG data revealed significant associations of haplotypes in certain SNP windows with mean BNEG % score changes in the African-American population—specifically, in SNP windows 3-4 (P=0.021), 4-5 (P=0.026), 3-4-5 (P=0.049). Table 3 shows the respective haplotypes significantly associated with mean BNEG % score changes. Haplotype 1-2 in SNP window 3-4, haplotype 2-1 in SNP window 4-5, and haplotype 1-2-1 in SNP window 3-4-5 were all significantly associated with increase in mean BNEG % score (P<0.05). On the other hand, haplotype 2-1 in SNP window 3-4, haplotype 1-1 in SNP window 4-5, and haplotype 2-1-1 in SNP window 3-4-5 were all significantly associated with decrease in mean BNEG % score (P<0.05).

TABLE 3
Haplotypes significantly associated with mean
BNEG % score changes in African-Americans

| SNP | Global | | Mean change | Chi-square | Haplotype |
|---|---|---|---|---|---|
| window | P-value | Haplotype | (n; freq) | (df) | P-value |
| 3-4 | 0.021 | 1-2 | 0.143 | 5.77 | 0.016 |
| | | | (19; 0.528) | (2) | |
| | | 2-1 | −0.341 | 6.42 | 0.011 |
| | | | (9; 0.250) | (2) | |
| 4-5 | 0.026 | 1-1 | −0.379 | 4.95 | 0.026 |
| | | | (7; 0.350) | (1) | |
| | | 2-1 | 0.107 | 4.95 | 0.026 |
| | | | (13; 0.650) | (1) | |
| 3-4-5 | 0.049 | 1-2-1 | 0.086 | 3.88 | 0.049 |
| | | | (11; 0.611) | (1) | |
| | | 2-1-1 | −0.379 | 3.88 | 0.049 |
| | | | (7; 0.389) | (1) | |

In Caucasians, significant association with increase in mean BNEG % score was found for haplotype 2-2-1 in SNP window 1-2-3 (P=0.005), as well as haplotype 2-1-1 in SNP window 2-3-4 (P=0.029). See Table 4 for details.

TABLE 4
Haplotypes significantly associated with
mean BNEG % score changes in Caucasians

| SNP | Global | | Mean change | Chi-square | Haplotype |
|---|---|---|---|---|---|
| window | P-value | Haplotype | (n; freq) | (df) | P-value |
| 1-2-3 | 0.020 | 2-2-1 | 0.889 | 7.80 | 0.005 |
| | | | (5; 0.156) | (2) | |
| 2-3-4 | 0.068 | 2-1-1 | 0.295 | 4.74 | 0.029 |
| | | | (16; 0.286) | (2) | |

For the total sample (Caucasians and African-Americans combined), haplotype 2-1-1 in SNP window 2-3-4 was significantly associated with increase in mean BNEG % score (P=0.011), while haplotype 1-1-1 in SNP window 1-2-3 was significantly associated with decrease in mean BPOS % score (P=0.038). See Table 5 for details.

TABLE 5
Haplotypes significantly associated with mean BPOS/BNEG %
score changes in Caucasians and African-Americans combined

| | SNP | Global | | Mean change | Chi-square | Haplotype |
|---|---|---|---|---|---|---|
| Scale | window | P-value | Haplotype | (n; freq) | (df) | P-value |
| BNEG | 1-2-3 | 0.054 | 2-2-1 | 0.592 | 6.51 | 0.011 |
| | | | | (10.2; 0.170) | (3) | |
| BPOS | 1-2-3 | 0.113 | 1-1-1 | −0.198 | 4.29 | 0.038 |
| | | | | (25.1; 0.419) | (3) | |

Percentage change in BPRS/BPOS/BNEG scores, rather than actual change in raw score, was used in the analysis because it was found that the amount of change in raw score is significantly associated with the baseline score.

We have identified significant association of SNP 5 (rs301434) with BPRS % score change in Caucasians (P=0.0125). On the other hand, SNP 1 (rs1980943) was significantly (P=0.0167) associated with BNEG % score change in African-Americans. These suggest that different SNPs in SLC1A1 influence clozapine treatment response in different ethnic groups.

For mean BNEG % score changes in the African-Americans, haplotype 1-2 in SNP window 3-4, haplotype 2-1 in SNP window 4-5, and haplotype 1-2-1 in SNP window 3-4-5 were all significantly associated with increase in mean BNEG % score (P<0.05). On the other hand, haplotype 2-1 in SNP window 3-4, haplotype 1-1 in SNP window 4-5, and haplotype 2-1-1 in SNP window 3-4-5 were all significantly associated with decrease in mean BNEG % score (P<0.05). These findings are especially robust, as these haplotypes are present in high frequencies. For instance, haplotypes 1-2-1 and 2-1-1 account for 100% of haplotypes found in SNP window 3-4-5 in African-Americans. These results suggest that the specific haplotypes in SNP window 3-4 significantly influence the direction of BNEG % score change in the African Americans.

Ethnic-specific haplotype association with BNEG % score change was also found in Caucasians—specifically, haplotype 2-2-1 in SNP window 1-2-3 (P=0.005) and haplotype 2-1.1 in SNP window 2-3-4 (P=0.029)—both associated with increase in BNEG % score i.e. worsening of negative symptoms.

In addition, several significant findings pertained to the entire population i.e. Caucasians and African-Americans combined. These include the associations of SNP 5 (P=0.049) and haplotype 2-1-1 in SNP window 2-3-4 (P=0.011) with mean BNEG % score change, as well as association of haplotype 1-1-1 in SNP window 1-2-3 with decrease in mean BPOS % score (P=0.038).

Taken together, these results suggest that different alleles and/or haplotypes of the SLC1A1 gene may predict the changes in positive and/or negative symptoms following clozapine response in either direction (increased or decreased) in Caucasians, African-Americans, or combined.

In particular, but not wishing to be bound by theory, the results of this study suggest a role of the glutamate transporter gene SLC1A1 in clozapine response in a treatment refractory/intolerant schizophrenic population. Using mean % change in BPRS/BPOS/BNEG scores, we have identified alleles and haplotypes in both Caucasians and African-Americans that have been significantly associated with increased or decreased BPRS/BPOS/BNEG—thus suggesting decreased or increased response to clozapine, respectively.

The present invention has been described with regard to one or more embodiments. However, it will be apparent to persons skilled in the art that a number of variations and modifications can be made without departing from the scope of the invention as defined in the claims.

### REFERENCES

Dudbridge F. Pedigree disequilibrium tests for multilocus haplotypes. Genetic Epidemiology. 2003; 25:115-21
Jentsch J D, Roth R H. The neuropsychopharmacology of phencyclidine: from NMDA receptor hypofunction to the dopamine hypothesis of schizophrenia. Neuropsychopharmacology. 1999; 20:201-25.
Kanai Y, Hediger M A. The glutamate/neutral amino acid transporter family SLC1: molecular, physiological and pharmacological aspects. Pflugers Arch. 2004; 447:469-79.
Kane J M, Honigfield G, Singer J, Meltzer H Y. Clozapine for the treatment-resistant schizophrenic: a double-blind comparison with chlorpromazine. Arch Gen Psychiatry. 1988; 45:789-96
Kim J S, Kornhuber H H, Schmid-Burgk W, Holzmuller B. Low cerebrospinal fluid glutamate in schizophrenic patients and a new hypothesis on schizophrenia. Neurosci Lett. 1980; 20:379-82.
Meltzer H Y. Treatment-resistant schizophrenia—the role of clozapine. Curr Med Res Opin. 1997; 14:1-20.
Schmitt A, Zink M, Fetroianu G, May B, Braus D F, Henn F A. Decreased gene expression of glial and neuronal glutamate transporters after chronic antipsychotic treatment in rat brain. Neurosci Lett. 2003; 347:81-4.
Seeman P, Lee T, Chau-Wong M, Wong K. Antipsychotic drug doses and neuroleptic/dopamine receptors. Nature. 1976; 261:717-9.
Seeman P, Weinshenker D, Quirion R, Srivastava L K, Bhardwaj S K, Grandy D K et al. Dopamine supersensitivity correlates with D2High states, implying many paths to psychosis. Proc Natl Acad Sci USA. 2005; 102:3513-8.
Shigeri Y, Seal R P, Shimamoto K. Molecular pharmacology of glutamate transporters, EAATs and VGLUTs. Brain Res Rev. 2004; 45:250-65.

All citations/references are hereby incorporated by reference.

1-20. (canceled)

21. A method for predicting a subject's response to antipsychotic drug treatment comprising,
a) obtaining a biological sample from the subject; and
b) determining the presence or absence of one or more polymorphisms in the SLC1A1 gene of the subject,
wherein the presence of said one or more polymorphisms indicates the subject's response to the antipsychotic drug treatment.

22. The method of claim 21, wherein the one or more polymorphisms comprise one or more polymorphisms as defined by SEQ ID NOs: 1-5 or a combination thereof.

23. The method of claim 21, wherein the one or more polymorphisms comprise one or more polymorphisms having a sequence that exhibits between about 90% and 100% sequence identity with SEQ ID NOs: 1-5 or a combination thereof.

24. The method of claim 21, wherein said response is the change in positive symptoms, negative symptoms, or both.

25. The method of claim 24, wherein the response is the change in positive symptoms.

26. The method of claim 24, wherein the response is the change in negative symptoms.

27. The method of claim 24, wherein the positive symptoms comprise one or more of delusions, hallucinations, disorganized speech, disorganized behavior, and catatonic behavior; and the negative symptoms comprise one or more symptoms that reflect a diminution or loss of normal function.

28. The method of claim 24, wherein said response is relative to a psychiatric rating scale.

29. The method of claim 28, wherein said psychiatric rating scale comprises the Brief Psychiatric rating scale (BPRS), the positive symptom subscale (BPOS), the negative symptom subscale (BNEG), or a combination thereof.

30. The method of claim 21, wherein the antipsychotic drug comprises a drug that affects dopamine signaling.

31. The method of claim 21, wherein the antipsychotic drug is clozapine, trifluoperazine, thioridazine, haloperidol, haloperidol decanoate, thiothixene, chlorpromazine, fluphenazine, loxapine, perphenazine, perphenazine decanoate, perphenazine-amitriptyline, acetophenazine, molindone, mesoridazine, fluphenazine decanoate, methotrimeprazine, risperidone, aripiprazole or a combination thereof.

32. The method of claim 21, wherein the subject is a Caucasian subject or an African-American subject.

33. The method of claim 21, wherein the subject exhibits psychotic symptoms, Schizophrenia symptoms, Schizoaffective disorder symptoms or a combination thereof.

34. The method of claim 33, wherein the subject is diagnosed as having a disorder with psychotic symptoms, Schizophrenia or Schizoaffective disorder.

35. The method of claim 21, wherein the sample is selected from the group consisting of blood, saliva, spinal fluid, brain biopsy, cultured cells obtained from the subject, stool, urine, autopsy samples, and frozen sections.

36. The method of claim 35, wherein the sample is blood.

37. The method of claim 21, wherein the step b) is achieved by PCR analysis, sequencing, 5′exonuclease fluorescence assay, probe hybridization or a combination thereof.

38. A method for predicting a subject's response to antipsychotic drug treatment comprising,
a) obtaining a biological sample from the subject; and
b) determining the presence or absence of one or more polymorphisms selected from the group consisting of SEQ ID NO:1, SEQ ID NO:2, SEQ ID NO:3, SEQ ID NO:4, SEQ ID NO:5, and any combination thereof, wherein,
the presence of the C allele of the rs301434 polymorphism (SEQ ID NO: 5) in Caucasians predicts improvement in overall psychosis symptoms;
the presence of the C allele of the rs301434 polymorphism (SEQ ID NO:5) in Caucasians and African-Americans combined predicts improvement in negative symptoms;
the G/A genotype of the rs1980943 polymorphism (SEQ ID NO:1) in African-Americans predicts improvement in negative symptoms;
the haplotype 1-2 in SNP window 3-4 (the G allele of the rs2228622 polymorphism (SEQ ID NO:3) in combination with the C allele of the rs301430 polymorphism (SEQ ID NO:4)); the haplotype 2-1 in SNP window 4-5 (the C allele of the rs301430 polymorphism (SEQ ID NO:4) in combination with the T allele of the rs301434 polymorphism(SEQ ID NO:5)), and haplotype 1-2-1 in SNP window 3-4-5 (the G allele of the rs2228622 polymorphism (SEQ ID NO:3) in combination with the C allele of the rs301430 polymorphism (SEQ ID NO:4) and the T allele of the rs301434 polymorphism (SEQ ID NO:5)) in African Americans are each associated with worsening of negative symptoms;
the haplotype 2-1 in SNP window 3-4 (the A allele of the rs2228622 polymorphism (SEQ ID NO:3) in combination with the T allele in the rs301430 polymorphism (SEQ ID NO:4)), haplotype 1-1 in SNP window 4-5 (the T allele in the rs301430 polymorphism (SEQ ID NO:4) in combination with the T allele in the rs301434 polymorphism (SEQ ID NO:5) and haplotype 2-1-1 in SNP window 3-4-5 (the A allele in the rs2228622 polymorphism (SEQ ED NO:3) in combination with the T allele in the rs301430 polymorphism (SEQ ID NO:4) and the T allele in the rs301434 polymorphism (SEQ ID NO:5)) in African Americans are each associated with improvement in negative symptoms;
the haplotype 2-2-1 in SNP window 1-2-3 (the A allele in the rs1980943 polymorphism (SEQ ID NO:1) in combination with the C allele in the rs3780415 polymorphism (SEQ ID NO:2) and the G allele in the rs2228622 polymorphism (SEQ ID NO:3)) and haplotype 2-1-1 in SNP window 2-3-4 (the C allele of rs3780415 (SEQ ID NO:2) in combination with the G allele of the rs2228622 polymorphism (SEQ ID NO:3) and the T allele of the rs301430 polymorphism (SEQ ID NO:4)) in Caucasians are both independently associated with worsening of negative symptoms;
the haplotype 2-2-1 in SNP window 1-2-3 (allele A of the rs1980943 polymorphism (SEQ ID NO:1) in combination with allele C of the rs3780415 polymorphism (SEQ ID NO:2) and allele G of the rs2228622 polymorphism (SEQ ID NO:3)) in both Caucasians and African Americans is associated with worsening of negative symptoms; and
the haplotype 1-1-1 in SNP window 1-2-3 (allele G of the rs1980943 polymorphism (SEQ ID NO:1) in combination with allele T of the rs3780415 polymorphism (SEQ ID NO;2) and allele G of the rs2228622 polymorphism (SEQ ID NO:3)) in both Caucasians and African Americans is associated with improvement of positive symptoms.

39. A kit comprising,
a) one or more primers to amplify a nucleotide sequence that comprises the polymorphism as defined in SEQ ID NOs: 1-5 or a combination thereof;
b) one or more probes that hybridize to SEQ ID NOs: 1-5, over a region of nucleotides comprising the polymorphic site, wherein said probe hybridizes to a particular variant of the polymorphism at the polymorphic site;
c) one or more reagents or products comprising buffers, nucleotides, DNA amplifying enzymes, or any combination thereof;
d) one or more reagents, components or products for genotyping the polymorphisms of SEQ ID NOs: 1-5, or a combination thereof;
e) one or more reagents, components or products for performing a DNA sequencing reaction that determines the sequence of SEQ ID NOs: 1-5, or a combination thereof;
f) one or more instructions for using the components as described herein, practicing the methods as described herein, interpreting the data obtained from practicing the methods, or any combination thereof;
g) a scale, reference or the like that may be used to test, diagnose, monitor or determine a baseline of symptoms for a subject, or
h) any combination or subcombination of a) through g).

40. The kit of claim 39, wherein the scale comprises the Brief Psychiatric Rating Scale (BPRS), positive symptom subscale (BPOS), negative symptom subscale (BNEG), or a combination thereof.


SEQUENCE LISTING (5 sequences)

SEQ ID NO: 1 - DNA, 51 bp, Artificial
  human SNP sequence rs1980943
agagtcagttggtgcatacatgtccngcatttgcaagagagggtctgtgtt

SEQ ID NO: 2 - DNA, 51 bp, Artificial
  human SNP sequence rs3780415
tgaggtgaaaaaagcaaataaatgcntttacctaaaataaagagctagcag

SEQ ID NO: 3 - DNA, 51 bp, Artificial
  human SNP sequence rs2228622
caggcagcacccctgaagtcagtacngtggatgccatgttagatctcatca

SEQ ID NO: 4 - DNA, 51 bp, Artificial
  human SNP sequence rs301430
gtgcaacaatcaacatggatgggacngcgctctatgaagcagtggcagcgg

SEQ ID NO: 5 - DNA, 51 bp, Artificial
  human SNP sequence rs301434
ggcaaggacttgtctccagaaagcantgtaggtgtggcctccagcttatcc